(19) United States
(12) Patent Application Publication
(10) Pub. No.: US 2026/0041934 A1
(43) Pub. Date: Feb. 12, 2026

(54) METHODS FOR DEFORMABLE IMAGE REGISTRATION OF MEDICAL IMAGES

(71) Applicant: Elekta Limited, Crawley, West Sussex (GB)

(72) Inventors: Wenzhe Yin, Crawley (GB); Jan-Jakob Sonke, Amsterdam (NL); Efstratios GAVVES, Crawley (GB)

(21) Appl. No.: 19/295,501

(22) Filed: Aug. 8, 2025

(30) Foreign Application Priority Data
Aug. 9, 2024 (GB) 2411754.1

(51) Int. Cl. A61N 5/10; G06T 7/00; G06T 7/33; G06T 7/38; G16H 30/20

(52) U.S. Cl. CPC A61N 5/1039; G06T 7/0016; G06T 7/344; G06T 7/38; G16H 30/20; G06T 2207/20036; G06T 2207/20081; G06T 2207/20084; G06T 2207/30048; G06T 2207/30241

(57) ABSTRACT

A method for performing deformable image registration of a first volumetric medical image to a second volumetric medical image may comprise estimating a time varying velocity field between the images by encoding coordinates using a time varying positional embedding and using the encoded coordinates and a Neural Field Ordinary Differential Equation (NFODE) to generate a prediction of the rate of change of the deformation field. The method may further comprise integrating the estimated velocity field to generate a deformation field and applying the generated deformation field to the first volumetric medical image to generate a registered volumetric medical image. The NFODE May comprise a non-stationary Neural ODE parameterized by an Implicit Neural Representation.

### CLAIM FOR PRIORITY

This application claims the benefit of priority of British Application No. 2411754.1, filed Aug. 9, 2024, which is hereby incorporated by reference in its entirety.

### TECHNICAL FIELD

The present disclosure relates to a method for performing deformable image registration of a first volumetric medical image to a second volumetric medical image. The method may be performed by a registration node. The present disclosure also relates to a registration node, and to a computer program product configured, when run on a computer, to carry out a method for performing deformable image registration of a first volumetric medical image to a second volumetric medical image.

### BACKGROUND

Deformable image registration is a fundamental task in computer vision, playing a critical role in various medical imaging analysis applications. This task aims to find a spatial mapping between moving and target images, so as to align them in a shared coordinate space. Some methods formulate deformable image registration as an optimization problem in which the transformation parameters are iteratively updated by minimizing a similarity function between the transformed moving image and the fixed target image.

### SUMMARY

With the advent of deep learning in medical image analysis, methods using either convolutional neural networks (CNNs) or transformer networks have demonstrated promising potential in deformable medical image registration. Several attempts have been made to improve the performance of these methods, including using multi-stage prediction and adversarial training. These approaches are trained on large, specialized datasets, a requirement that poses two significant challenges. Firstly, learned features are sensitive to data distribution change, such as will occur with a change in imaging modality, for example changing from Computed Tomography (CT) to Magnetic Resonance Imaging (MRI). Secondly, a learned feature cannot accurately describe the geometric transformation between all image pairs in the dataset. Attempts have been made to address these challenges by adopting test-time optimization. However, these approaches need to finetune or optimize a large network at test time, leading to lower computation efficiency.

Another research approach in deformable image registration is based on the recent progress in Implicit Neural Representations (INRs). INRs are continuous neural field functions that map each coordinate in a space to desired local properties. In the application of image registration, an INR can be used to approximate a deformation field. Implicit Deformable Image Registration (IDIR) is an INR approach to deformable image registration proposed by Wolterink, J. M., Zwienenberg, J. C., Brune, C. in “Implicit neural representations for deformable image registration”: International Conference on Medical Imaging with Deep Learning. pp. 1349-1359. PMLR (2022). In the proposed IDIR approach, the INR is parameterized as a SIREN network, which is a multilayer perceptron (MLP) with sinusoidal activation functions. In order to achieve reliable deformable registration, suitable regularization techniques are necessary to ensure that the estimated deformation field between images is smooth and realistic. In terms of INR based registration, IDIR benefits from the high-order differentiable property of SIREN, and introduces a bending energy constraint in the form of second order derivatives. Other regularization techniques like cycle consistency and conformal-invariant hyperelastic regularization have been proposed for further improved registration.

Although IDIR based methods have achieved promising performance on image registration, they are limited by the capacity of SIREN which, as an MLP, requires a large capacity to model complex signals.

It is an aim of the present disclosure to provide a method, a registration node, and a computer program product which at least partially address one or more of the challenges mentioned above. It is a further aim of the present disclosure to provide a method, a registration node, and a computer program product which cooperate to perform deformable image registration of volumetric medical images that achieves improved performance when compared with existing methods.

According to a first aspect of the present disclosure, there is provided a computer implemented method for performing deformable image registration of a first volumetric medical image, associated with a first time instance, to a second volumetric medical image, associated with a second time instance. The method can comprise estimating a time varying velocity field between the first and second volumetric medical images by performing a series of steps for positions within the first volumetric medical image, and for time instances between the first time instance and the second time instance. The operations can include encoding coordinates of the position using a time varying positional embedding, and using the encoded coordinates and a Neural Field Ordinary Differential Equation (NFODE) to generate a prediction of the rate of change of the deformation field from the first volumetric medical image to the second volumetric medical image. The method can further comprise integrating the estimated velocity field between the first and second time instances to generate a deformation field from the first volumetric medical image to the second volumetric medical image, and applying the generated deformation field to the first volumetric medical image to generate a registered volumetric medical image. For the purposes of the method, an NFODE can comprise a non-stationary Neural ODE that is parameterized by an Implicit Neural Representation.

According to another aspect of the present disclosure, there is provided a computer implemented method for adaptation of a reference Radiotherapy (RT) treatment plan, wherein the reference RT treatment plan is associated with a first volumetric medical image of a patient. The method can comprise acquiring a second volumetric medical image of a patient, performing deformable image registration of the first volumetric medical image to the second volumetric medical image using a method according to any one of the aspects or examples of the present disclosure, and using the generated deformation field between the first and second volumetric medical images to adapt the reference treatment plan.

According to another aspect of the present disclosure, there is provided a computer program product comprising a computer readable non-transitory medium, the computer readable medium having computer readable code embodied therein, the computer readable code being configured such that, on execution by a suitable computer or processor, the computer or processor is caused to perform a method according to any one of the aspects or examples of the present disclosure.

According to another aspect of the present disclosure, there is provided a registration node for performing deformable image registration of a first volumetric medical image, associated with a first time instance, to a second volumetric medical image, associated with a second time instance. The registration node can comprise processing circuitry configured to cause the registration node to estimate a time varying velocity field between the first and second volumetric medical images by performing a series of steps for positions within the first volumetric medical image, and for time instances between the first time instance and the second time instance. The operations can include encoding coordinates of the position using a time varying positional embedding, and using the encoded coordinates and a Neural Field Ordinary Differential Equation (NFODE) to generate a prediction of the rate of change of the deformation field from the first volumetric medical image to the second volumetric medical image. The processing circuitry can further configured to cause the registration node to integrate the estimated velocity field between the first and second time instances to generate a deformation field from the first volumetric medical image to the second volumetric medical image, and apply the generated deformation field to the first volumetric medical image to generate a registered volumetric medical image. An NFODE comprises a non-stationary Neural ODE that is parameterized by an Implicit Neural Representation.

According to another aspect of the present disclosure, there is provided radiotherapy treatment apparatus comprising a registration node according to any one of the aspects or examples of the present disclosure.

According to another aspect of the present disclosure, there is provided planning node for adapting a reference Radiotherapy (RT) treatment plan, wherein the reference RT treatment plan is associated with a first volumetric medical image of a patient. The planning node can comprise processing circuitry configured to cause the planning node to acquire a second volumetric medical image of a patient, and to perform deformable image registration of the first volumetric medical image to the second volumetric medical image using a method according to any one of the aspects or examples of the present disclosure. The processing circuitry can further be configured to cause the planning node to use the generated deformation field between the first and second volumetric medical images to adapt the reference treatment plan.

According to another aspect of the present disclosure, there is provided radiotherapy treatment apparatus comprising a planning node according to any one of the aspects or examples of the present disclosure.

Aspects of the present disclosure thus provide a method and registration node that use a Neural Field to model a velocity field for deformable image registration. Methods according to the present disclosure allow for an efficient increase in capacity for modeling complex deformation, as well as alleviating the reliance on sophisticated bending energy regularization in known IDR based registration methods.

### BRIEF DESCRIPTION OF THE DRAWINGS

For a better understanding of the present disclosure, and to show more clearly how it may be carried into effect, reference will now be made, by way of example, to the following drawings in which:

FIG. 1 is an example of a flow chart illustrating process steps in a computer implemented method for performing deformable image registration of a first volumetric medical image to a second volumetric medical image;

FIGS. 2a to 2d are examples of flow charts illustrating another example of a method for performing deformable image registration of a first volumetric medical image to a second volumetric medical image;

FIG. 3 is an example of a block diagram illustrating functional modules in an example registration node;

FIG. 4 illustrates an example implementation framework for the methods disclosed herein;

FIG. 5 presents a table of experimental results;

FIG. 6 presents visualisations of some of the experimental results illustrated in FIG. 5;

FIG. 7 presents another table of experimental results; and

FIG. 8 presents another table of experimental results.

### DETAILED DESCRIPTION

Examples of the present disclosure propose the use of a Neural Field Ordinary Differential Equation (NFODE) for medical image registration. The proposed NFODE parameterizes a non-stationary Neural ODE using an Implicit Neural Representation (INR). As discussed in greater detail below, the ODE proposed herein offers properties including diffeomorphism and non-intersecting trajectory which facilitate implicit regularization on the deformation field. Consequently, the methods proposed herein achieve improved registration without any additional explicit regularization. In addition, in some examples, the methods proposed herein can incorporate a total derivative regularization substantially seamlessly, so as to encourage straight-line trajectories in a similar manner to the optimal transport cost regularization used in Finlay, C., Jacobsen, J. H., Nurbekyan, L., Oberman, A., “How to train your neural ode: the world of jacobian and kinetic regularization”, in International conference on machine learning. pp. 3154-3164. PMLR (2020).

Owing to the large degree of freedom in the deformable registration task, it may be beneficial to learn the deformation from coarse to fine (global rigid motion to local non-rigid local). Such progression implies a non-stationary ODE, and the present disclosure introduces two features related to this. Firstly, a time-varying frequency position encoding scheme is proposed, allowing the ODE to learn to deform from coarse to fine. Secondly, a time-varying residual weight is proposed in the context of the Neural ODE. For the time-varying frequency position encoding, the low and high frequencies of sinusoidal function in the implicit neural field positional embedding produce relative global and local signals, respectively, hence enabling deformation from coarse to fine. The time-varying residual weight provides a way to model the time-varying ODE function, and so increase the model capacity of SIREN, enabling the SIREN MLP to capture complex signals. As discussed in greater detail below, in a departure from previous approaches to INR based registration, examples of the present disclosure use a continuous INR as ODE function which is flexible on different data. In addition, the present disclosure proposes to model the non-stationary velocity fields, implicitly inducing a more flexible and larger modeling capacity.

Examples of the present disclosure thus propose an implicit neural field-parameterized ODE for deformable registration. As demonstrated in the experimental results presented below, the proposed NFODE outperforms the baseline IDIR model without requiring additional explicit regularization. The proposed NFODE uses a time-varying frequency scheme of position encoding from the low frequency to high frequency to learn the deformation from coarse to fine. In addition, a time-varying weights scheme may be used to make the NFODE non-stationary, leading to larger model capacity and better flexibility. In further examples, a total derivative regularization is introduced for smoother trajectories.

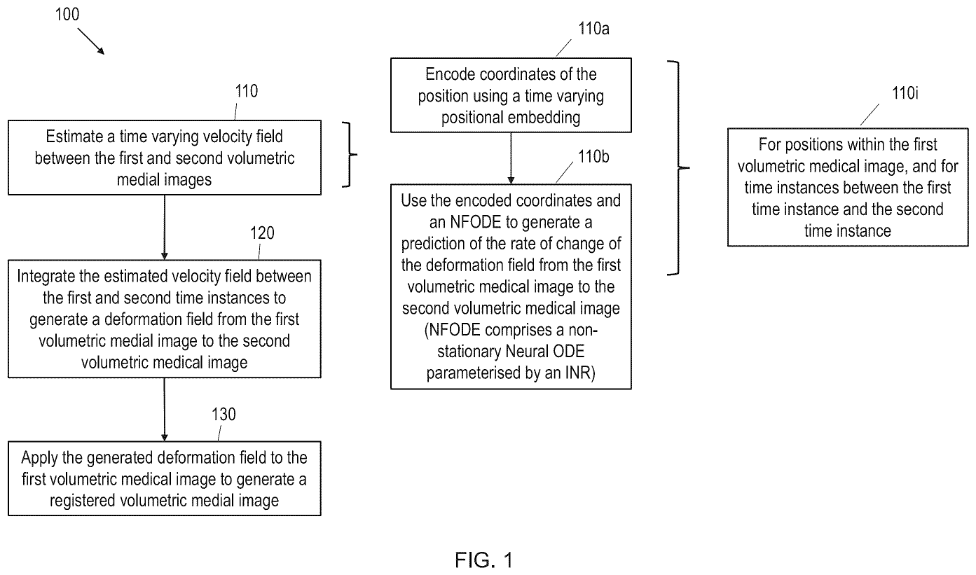

FIG. 1 is a flow chart illustrating process steps in a computer implemented method 100 for performing deformable image registration of a first volumetric medical image, associated with a first time instance, to a second volumetric medical image, associated with a second time instance. The method may be performed by a registration node, which may comprise a physical or virtual node, and may be implemented in a computer system, treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The registration node may encompass multiple logical entities, as discussed in greater detail below.

Referring to FIG. 1, the method 100 comprises, in a first step 110, estimating a time varying velocity field between the first and second volumetric medical images. Step 110 is performed by executing steps 110a and 110b for positions within the first volumetric medical image, and for time instances between the first time instance and the second time instance, as illustrated at 110i. In step 110a, the method comprises encoding coordinates of the position using a time varying positional embedding. In step 110b, the method comprises using the encoded coordinates and a Neural Field Ordinary Differential Equation (NFODE) to generate a prediction of the rate of change of the deformation field from the first volumetric medical image to the second volumetric medical image. Following estimation of the time varying velocity field in step 110, the method then comprises, in step 120, integrating the estimated velocity field between the first and second time instances to generate a deformation field from the first volumetric medical image to the second volumetric medical image. In step 130, the method comprises applying the generated deformation field to the first volumetric medical image to generate a registered volumetric medical image. As illustrated at step 110b, an NFODE, as used in step 110b, comprises a non-stationary Neural ODE that is parameterized by an Implicit Neural Representation (INR).

It will be appreciated that according to examples of the present disclosure, the first and second time instances are the beginning and end of a predetermined time period running between the first and second medical images. It will further be appreciated that that these time instances do not have to be times at which the images were captured. The time period defined by the first and second time instances may be an introduced, imaginary time period which may for example be set to run from 0 to 1.

As discussed above, an INR, or Neural Field, is a neural architecture that parameterizes a field, i.e., a quantity defined over spatial and/or temporal coordinates, using a neural network. An INR may thus comprise, for example, the values of trained parameters of the neural network that parameterizes the field, including for example the weights and biases of the neural network. A neural network is an example of a Machine Learning (ML) model. For the purposes of the present disclosure, the term “ML model” encompasses within its scope the following concepts:
machine Learning algorithms, comprising processes or instructions through which data may be used in a training process to generate a model artefact for performing a given task, or for representing a real-world process or system; and
the model artefact that is created by such a training process, and which comprises the computational architecture that performs the task.

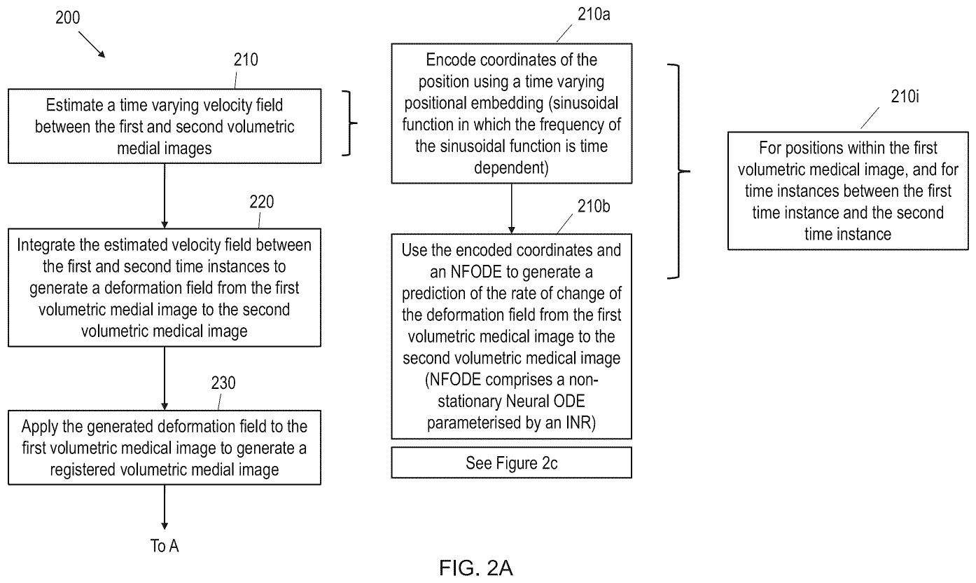

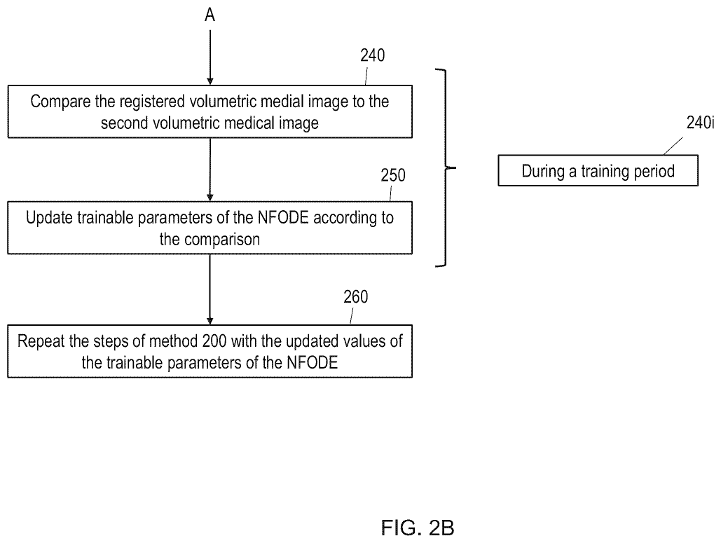

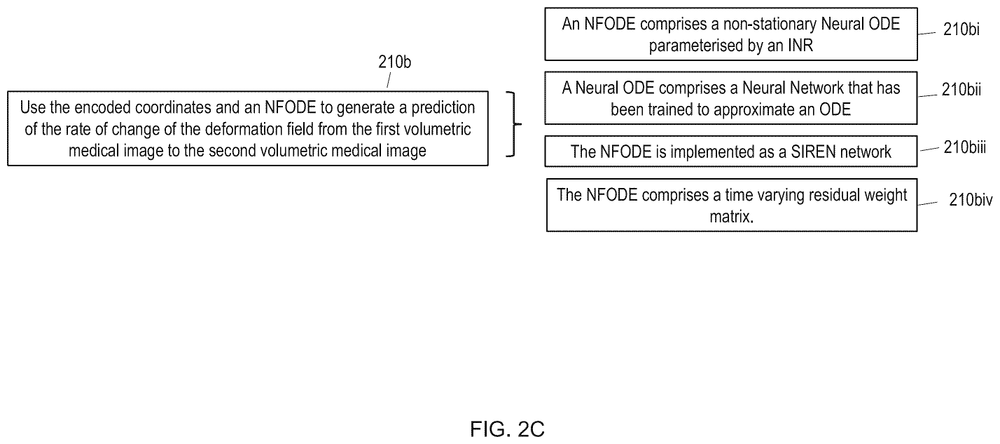

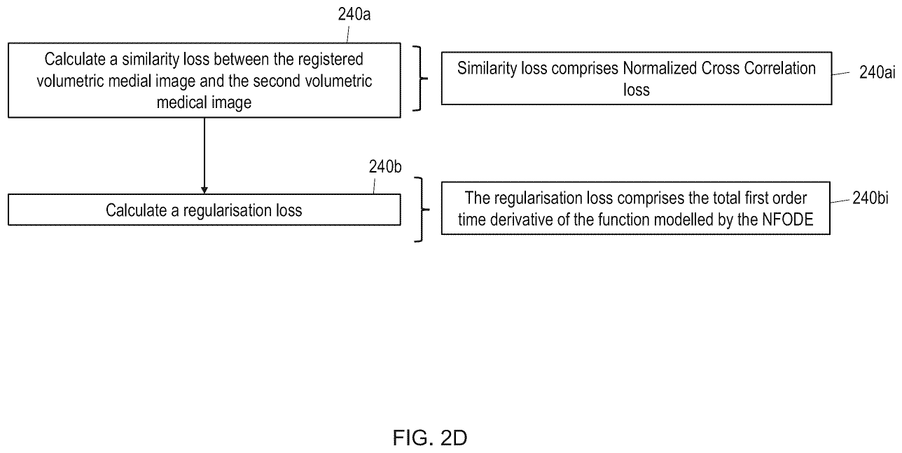

FIGS. 2a to 2d show flow charts illustrating another example of a method 200 for performing deformable image registration of a first volumetric medical image, associated with a first time instance, to a second volumetric medical image, associated with a second time instance. As for the method 100 discussed above, the method 200 may be performed by a registration node, which may comprise a physical or virtual node, and may be implemented in a computer system, treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The registration node may encompass multiple logical entities, as discussed in greater detail below. The method 200 illustrates an example of how the steps of the method 100 may be implemented and supplemented to provide the above discussed and additional functionality.

Referring initially to FIG. 2a, in step 210, the registration node estimates a time varying velocity field between the first and second volumetric medical images. As illustrated at 210i, the registration node performs this estimation by carrying out steps 210a and 210b for positions within the first volumetric medical image, and for time instances between the first time instance and the second time instance.

Step 210a comprises encoding coordinates of the position using a time varying positional embedding. In some examples, as illustrated at 210b, the time varying positional embedding may comprise a sinusoidal function in which the frequency of the sinusoidal function is time dependent. The time dependent frequency may for example vary from low frequency to high frequency with increasing time. It will be appreciated that low and high frequencies of sinusoidal function in implicit neural field positional encoding produce global and local signals respectively, allowing the NFODE in the next step to learn to deform from coarse to fine.

In some examples, the time varying positional embedding may comprise:

$$Γ ( t , p ) = [ sin ( B ( t ) p ) , cos ( B ( t ) p ) ]$$

where: Γ(t, p)∈ [symbol] ⁶ is a time dependent position encoding function
   B(t)=2^{-α+βt} controls the frequency of sinusoidal function
   α and β are hyperparameters

Following the positional embedding of step 210a, for positions within the first volumetric medical image, and for time instances between the first time instance and the second time instance, estimating the velocity field in step 210 comprises performing step 210b. Step 210b comprises using the encoded coordinates and a Neural Field Ordinary Differential Equation (NFODE), to generate a prediction of the rate of change of the deformation field from the first volumetric medical image to the second volumetric medical image.

Further detail that may be include din step 210b is illustrated in FIG. 2c. Referring now to FIG. 2c, and as discussed above and illustrated at 210bi, for the purposes of the methods 100 and 200, an NFODE comprises a non-stationary Neural ODE that is parameterized by an INR. As illustrated at 210bii, a Neural ODE comprises a Neural Network that has been trained to approximate an Ordinary Differential Equation. In some examples, a Neural ODE may consequently be trained using an ODE numerical solver, so as to result in a trained Neural ODE that replicates the performance of the ODE numerical solver.

As illustrated at 210biii, the NFODE may be implemented as a SIREN network, which is a Multilayer Perceptron (MLP) that uses the sine function as activation function. As illustrated at 210biv, the NFODE may comprise a time varying residual weight matrix. This time varying residual weight matrix may enable modelling of a time varying ODE function, and so increase the model capacity of SIREN, allowing for capturing of complex signals such as the velocity field between the first and second images.

In some examples, the NFODE may implement:

$$f ( {h}_{i} ) = {σ}_{i} ( {W}_{i} ( t ) {h}_{i} + {b}_{i} ( t ) )$$

$${W}_{i} ( t ) = {W}_{i} + {\sum }_{r = 1}^{{R}_{i}} {c}_{i} ( t ) [ r ] \cdot {M}_{i} [ r ]$$

where: hᵢ∈ [symbol] ^{M}^{ᵢ} is the hidden states at layer i
   σᵢ is the sinusoidal activation function
   Wᵢ(t) is the time varying residual weight matrix at layer i
   bᵢ∈ [symbol] ^{N}^{₁} is the basis vector at layer i
   c(t)∈ [symbol] ^{R}^{ᵢ} are trainable coefficients
   M∈ [symbol] ^{R}^{ᵢ}^{×N}^{ᵢ}^{×M}^{ᵢ} is the spanning basis set

Referring again to FIG. 2a, following estimation of the time varying velocity field between the first and second volumetric medical images in step 210, the registration node then, in step 220, integrates the estimated velocity field between the first and second time instances to generate a deformation field from the first volumetric medical image to the second volumetric medical image. In step 230, the registration node then applies the generated deformation field to the first volumetric medical image to generate a registered volumetric medical image.

Referring now to FIG. 2b, in some examples of the present disclosure, the method 200 may further comprise performing steps 240 and 250 during a training period, as illustrated at step 240i. In step 240, the registration node compares the registered volumetric medical image to the second volumetric medical image. In step 250, the registration node updates trainable parameters of the NFODE according to the comparison. In some examples, the registration node may for example us the Adam optimizer to update the trainable parameters of the NFODE.

Additional sub steps that may be carried out in order to perform the comparison at step 240 are illustrated in FIG. 2d.

Referring now to FIG. 2d, in a first sub step 240a, the registration node calculates a similarity loss between the registered volumetric medical image and the second volumetric medical image. As illustrated at 240ai, the similarity loss may comprise Normalized Cross Correlation (NCC) loss. In a second sub step 240b, the registration node then calculates a regularisation loss. As illustrated at 240bi, the regularisation loss may comprise the total first order time derivative of the function modelled by the NFODE. For example, the regularisation loss may comprise:

$${L}_{r e g} = {\int }_{0}^{1} {‖ \frac{\partial f ( ϕ , t )}{\partial ϕ} f ( ϕ , t ) + \frac{\partial f ( ϕ , t )}{\partial t} ‖}_{2}^{2} dt$$

Referring again to FIG. 2b, following updating of the trainable parameters in step 250, the registration node may return to step 210 and repeat the steps of the method 200 with the updated values of the trainable parameters of the NFODE.

Example methods according to the present disclosure achieve deformable image registration that offers both speed and accuracy. The methods described above offer the speed advantages associated with ML solutions when compared with classical procedures. In addition, the methods proposed herein offer improved accuracy compared with existing INR based methods, as is demonstrated in the experimental data presented below. This combination of speed and accuracy can support greater speed in both the planning and delivery of radiotherapy treatment.

The speed and accuracy afforded by methods of the present disclosure can support real-time or near real-time scenarios and applications for deformable image registration. The technical benefits of this provision include reduced radiotherapy treatment plan creation time, and may result in many additional medical treatment benefits (including improved accuracy of radiotherapy treatment, reduced exposure to unintended radiation, reduced treatment duration, etc.). The methods presented herein may be applicable to a variety of medical treatment and diagnostic settings or radiotherapy treatment equipment and devices.

In one particular use case for methods of the present disclosure, a dose from a previous treatment session can be deformed or modified in light of the generated deformation field and registered volumetric medical image. By determining the accurate mapping of voxels from one image to another, a determination can be made as to the amount of dose delivered to a particular target depicted in the images and/or the amount of movement of the target between the times at which the images were taken. Based on the amount of delivered dose and/or movement of the target, the dose can be deformed. The output of the methods disclosed herein may thus be used in the creation or adaptation of a radiotherapy treatment plan.

Examples of the present disclosure also propose a computer implemented method for adaptation of a reference radiotherapy treatment plan, wherein the reference radiotherapy treatment plan is associated with a first volumetric medical image of a patient. The method comprises acquiring a second volumetric medical image of a patient, performing deformable image registration of the first volumetric medical image to the second volumetric medical image using a method according to any one or more of the examples described herein, and using the generated deformation field between the first and second volumetric medical images to adapt the reference radiotherapy treatment plan.

Examples of the present disclosure also propose a planning node for adapting a reference radiotherapy treatment plan, wherein the reference radiotherapy treatment plan is associated with a first volumetric medical image of a patient. The planning node comprises processing circuitry configured to cause the planning node execute the above discussed method.

Examples of the present disclosure also propose a radiotherapy treatment apparatus comprising a planning node as set out above.

As discussed above, the methods 100 and 200 may be performed by a registration node, and the present disclosure provides a registration node that is adapted to perform any or all of the steps of the above discussed methods. The registration node may comprise a physical or virtual node, and may be implemented in a computer system, treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The registration node may encompass multiple logical entities, as discussed in greater detail below.

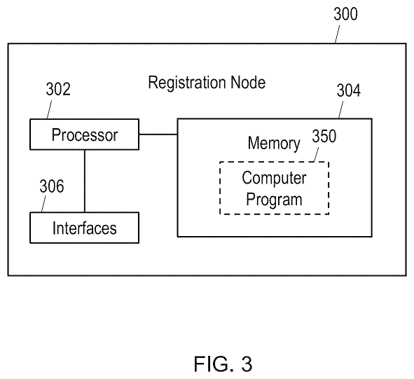

FIG. 3 is a block diagram illustrating an example registration node 300 which may implement the method 100 and/or 200, as illustrated in FIGS. 1 and 2a to 2d, according to examples of the present disclosure, for example on receipt of suitable instructions from a computer program 350. Referring to FIG. 3 the registration node 300 comprises a processor or processing circuitry 302, and may comprise a memory 304 and interfaces 306. The processing circuitry 302 is operable to perform some or all of the steps of the method 100 and/or 200 as discussed above with reference to FIGS. 1 and 2a to 2d. The memory 304 may contain instructions executable by the processing circuitry 302 such that the registration node 300 is operable to perform some or all of the steps of the method 100 and/or 200, as illustrated in FIGS. 1 and 2a to 2d. The instructions may also include instructions for executing one or more telecommunications and/or data communications protocols. The instructions may be stored in the form of the computer program 350. In some examples, the processor or processing circuitry 302 may include one or more microprocessors or microcontrollers, as well as other digital hardware, which may include digital signal processors (DSPs), special-purpose digital logic, etc. The processor or processing circuitry 302 may be implemented by any type of integrated circuit, such as an Application Specific Integrated Circuit (ASIC), Field Programmable Gate Array (FPGA) etc. The memory 304 may include one or several types of memory suitable for the processor, such as read-only memory (ROM), random-access memory, cache memory, flash memory devices, optical storage devices, solid state disk, hard disk drive, etc.

In some examples as discussed above, the registration node may be incorporated into treatment apparatus, and examples of the present disclosure also provide a treatment apparatus, such as a radiotherapy treatment apparatus, comprising either or both of a registration node as discussed above and/or a planning node operable to implement a method for adapting a radiotherapy treatment plan, also as discussed above.

FIGS. 1 to 2d discussed above provide an overview of methods which may be performed according to different examples of the present disclosure. These methods may be performed by a registration node, as illustrated in FIG. 3.

There now follows a detailed discussion of theoretical support for methods according to the present disclosure, as well as a description of how different process steps illustrated in FIGS. 1 to 2d and discussed above may be implemented. Also presented are experimental results for an example implementation. The functionality and implementation detail described below is discussed with reference to the modules of FIG. 3 performing examples of the methods 100 and/or 200, substantially as described above.

As discussed above, image registration addresses the situation of an image pair comprising a moving image Iₘ and a fixed image I_{f}. Image registration aims to find a transformation ϕ that minimizes a similarity function between the transformed image and the fixed image with regularization on the smoothness of the deformation vector field:

$${min}_{ϕ} L , L = {L}_{s i m} ( {I}_{m} ∘ ϕ , {I}_{f} ) + ( ϕ ) \qquad Equation l$$

where Lₛᵢₘ is the similarity loss, [symbol] is a regularization term on the deformation vector field ϕ, and Iₘ∘ϕ is the transformed moving image.

The methods according to the present disclosure model ODE-based registration by a time-dependent implicit neural network f(ϕ(t), t), with t∈[0, 1] introduced as the imaginary time variable running between the moving and the target image.

$$\frac{d ϕ ( t )}{d t} = f ( ϕ ( t ) , t ) ϕ ( 0 ) = p \qquad Equation 2$$

where p=(x, y, z)∈ [symbol] ³ is the initial position coordinate from a 3D medical image to be registered. Specifically, the learned Neural ODE function outputs f(ϕ(t), t) as the velocity field of the deformation at time t. To obtain the final deformation field the methods disclosed herein integrate with respect to t from 0 to 1. In each step, the ODE function outputs the velocity field f(ϕ(t), t), and once the deformation field has been obtained from the velocity field, and used to transform the moving image, it is then possible to measure the similarity between the time-evolved moving image Iₘ∘ϕ(1) and the final target image I_{f}. It will be appreciated that to solve the ODE numerically, time interval [0, 1] is discretized to t′=0, 1, . . . , Tsteps. With the ODE modeling, the solution presented herein is diffeomorphic, and the trajectory is free of self-intersection. The ODE parameterization consequently acts as an implicit regularization for smoothness in the integrated deformation field.

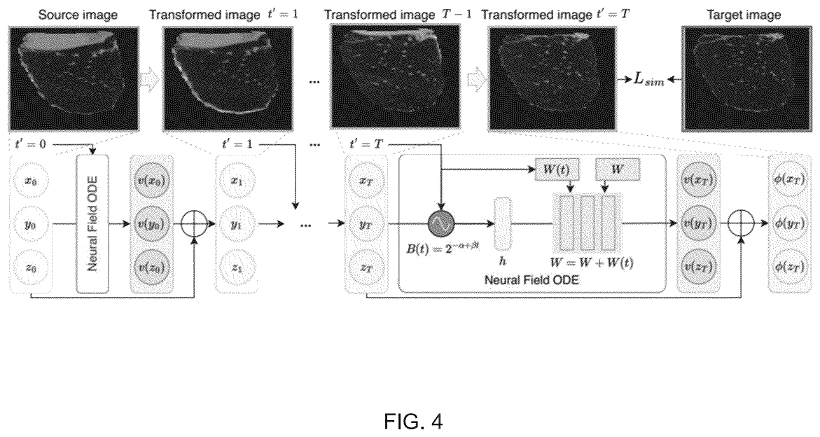

FIG. 4 illustrates an example implementation framework for the methods disclosed herein, showing the discretized ODE step t′=0, 1, . . . , T. First, a time-varying positional embedding is used to encode the position coordinate (steps 110, 210 of methods 100, 200). In each ODE step, the input is encoded by the time-varying position encoding (frequency changing with B (t)=2^{-α+βt}). Following this step, the time-dependent position embedding is passed into a Neural ODE (steps 120, 220 of methods 100, 200), which models how the deformation field evolves through time so that the moving image matches the target image. The Neural ODE is implemented as a SIREN network with sinusoidal nonlinearities. Further, to account for non-stationarity and to increase model flexibility, a time-varying residual weight matrix is introduced so that the ODE function becomes time-dependent (step 210biv of method 200). Additionally, a total derivative regularization is used to encourage smoother ODE trajectories (steps 240b and 240bi of method 200). The time-dependent ODE function (W+W (t)) predicts the velocity field. The deformation field ϕ is obtained by integrating the velocity field from [0, 1] (T discretized steps), and is used to transform the source image for evaluating similarity loss with the target image.

### Time-Varying Positional Encodings (TPE) (Steps 110, 210 of Methods 100, 200)

Previous works, including Hertz, A., Perel, O., Giryes, R., Sorkine-Hornung, O., Cohen-Or, D.: “Sape: Spatially-adaptive progressive encoding for neural optimization.” Advances in Neural Information Processing Systems 34, 8820-8832 (2021), have shown that the low and high frequencies of the sinusoidal function in the implicit neural field positional embedding produce relatively global and local signals, respectively. Examples of the present disclosure also seek to model deformation fields over time, where the initial time steps correspond to coarser fields that become finer and finer as time evolves. Consequently, examples of the present disclosure define spatiotemporal positional embeddings also with sinusoidal functions for NODE, that is

$$Γ ( t , p ) = [ sin ( B ( t ) p ) , cos ( B ( t ) p ) ] \qquad Equation 3$$

where: Γ(t, p)∈ [symbol] ⁶ is a time dependent position encoding function
   B(t)=2^{-α+βt} controls the frequency of sinusoidal function
   α and β are hyperparameters
   t∈[0,1]

The hyperparameters α and β control the starting lowest frequency and time-varying ratio, respectively. With evolving time, the frequency in the spatiotemporal positional embeddings changes from the low frequency to the high frequency to learn the motion from coarse to fine.

### Time-Varying INR (Step 210biv of Method 200)

For modeling the evolution of the deformation field with Neural ODE, first a stationary velocity function may be defined:

$$f ( {h}_{i} ) = {σ}_{i} ( {W}_{i} {h}_{i} + {b}_{i} ) \qquad Equation 4$$

where σᵢ is the sinusoidal activation function, Wᵢ∈ [symbol] ^{N}^{ᵢ}^{×M}^{ᵢ} and bᵢ∈ [symbol] R^{N}^{ᵢ} are MLP weight matrix and basis vector at layer i, respectively. hᵢ∈ [symbol] ^{M}^{ᵢ} is the hidden states at layer i. The velocity function is stationary in that the weights do not change with time. However, with complex and long deformations, stationarity is not necessarily guaranteed. For instance, the internal body movements might not conform to the same types of deformations through time. Some organs are softer than others and thus will have much smoother and more gradual deformations, while deformations around hard structures like bones are expected to be more intense. Parameterizing the Neural ODE to be able to model non-stationary time evolutions of the deformation field is therefore anticipated to provide additional advantages. To this end, in the presently described implementation, the parameters Wᵢ of the velocity function (Equation 4) are set to be to be time-dependent, that is:

$$f ( {h}_{i} ) = {σ}_{i} ( {W}_{i} ( t ) {h}_{i} + {b}_{i} ( t ) ) \qquad Equation 5$$

$${W}_{i} ( t ) = {W}_{i} + {\sum }_{r = 1}^{{R}_{i}} {c}_{i} ( t ) [ r ] \cdot {M}_{i} [ r ] \qquad Equation 6$$

where: hᵢ∈ [symbol] ^{M}^{ᵢ} is the hidden states at layer i
   σᵢ is the sinusoidal activation function
   Wᵢ(t) is the time varying residual weight matrix at layer i
   bᵢ∈ [symbol] ^{N}^{ᵢ} is the basis vector at layer i
   c(t)∈ [symbol] ^{R}^{ᵢ} are trainable coefficients
   M∈ [symbol] ^{R}^{ᵢ}^{×N}^{ᵢ}^{×M}^{ᵢ} is the spanning basis set

It will be appreciated that in this formulation, weight parameters are factorized, as proposed in Mihajlovic, M., Prokudin, S., Pollefeys, M., Tang, S.: “Resfields: Residual neural fields for spatiotemporal signals”. arXiv preprint arXiv: 2309.03160 (2023), allowing for memory efficient interpolating of time-dependent residual weights. Mi is shared for all time steps. The coefficients c(t) are initialized at T′ regular time steps and linearly interpolated to arbitrary time steps. It will be appreciated that in contrast to Mihajlovic et al., the present disclosure does not propose a sequence of individual INRs. Instead, Wᵢ is shared across time steps, which allows for a consistent ODE trajectory through time between two image pairs.

Another perspective on having time-varying parameterizations of Neural ODEs is the increased modeling complexity. It has been proven in Mihajlovic et al. that adding residual weights is more efficient and effective in modeling complex signals than simply increasing the MLP size.

### Similarity Loss (Steps 240a, 240Ai of Method 200)

The example implementation presented herein uses the normalized cross-correlation (NCC) between sampled intensities in the fixed image and corresponding intensities in the moving image to supervise the training.

### Total Derivative Regularization (Steps 240b and 240bi of Method 200)

The NFODE formulation can achieve excellent performance without additional regularization. Further improvement can be obtained by introducing total derivative regularization to encourage straight ODE trajectories. This can be achieved by regularizing the total time derivative of f which can be interpreted as a force acting on the trajectory. Regularizing the force over time then encourages straight-line trajectories. It will be appreciated that the total derivative regularization presented herein is based on the first derivative which is more efficient to compute than the second-order bending energy constraints used in IDIR as presented in the Background section.

$$\frac{d f ( ϕ , t )}{d t} = \frac{\partial f ( ϕ , t )}{\partial ϕ} \frac{d ϕ}{dt} + \frac{\partial f ( ϕ , t )}{\partial t} = \frac{\partial f ( ϕ , t )}{\partial ϕ} f ( ϕ , t ) + \frac{\partial f ( ϕ , t )}{\partial t} \qquad Equation 7$$

$${L}_{r e g} = {\int }_{0}^{1} {‖ \frac{\partial f ( ϕ , t )}{\partial ϕ} f ( ϕ , t ) + \frac{\partial f ( ϕ , t )}{\partial t} ‖}_{2}^{2} dt \qquad Equation 8$$

### Experiments

There now follows a presentation of experimental results obtained using the example implementation discussed above.

Dataset. The example implementation was evaluated using the DIR-LAB dataset presented in Castillo, R., Castillo, E., Guerra, R., Johnson, V. E., McPhail, T., Garg, A. K., Guerrero, T.: “A framework for evaluation of deformable image registration spatial accuracy using large landmark point sets”. Physics in Medicine & Biology 54(7), 1849 (2009), which is a standard benchmark for deformable registration. The dataset consists of ten 4D CT images with 300 manually labeled anatomical landmarks for evaluation. Images have in-plane resolutions from 256×256 pixel to 512×512 with various numbers of slices along the z dimension. The objective is to register the initial inspiration images to expiration images. The dataset presents a significant challenge for image registration owing to the large deformation of lung breathing and the complex interplay of cardiac and respiratory motions.

Evaluation Metric. Given the 300 predefined anatomical landmarks per CT scan pair, performance was evaluated by the target registration error (TRE). TRE measures the point-wise distance after registration.

Implementation details. The model was trained for 2500 epochs, and in each epoch, 10,000 points were randomly sampled. The Adam optimizer was adopted with a learning rate of 5e-4. All experiments were conducted on an NVIDIA GTX 1080Ti GPU. The model architecture contained a 4-layer MLP with sinusoidal activation functions and Rᵢ=spanning basis for each layer. The total derivative regularization was implemented by the PyTorch auto differentiation library. The weight for L_{reg} was 0.01 when used. For the time and performance tradeoff, the Euler method was adopted with a step size of 0.1, which gives T=10 time steps. To implement the time-varying positional encodings, values of α=8, and β=1 were chosen for scheduling the frequency through time. To enable a fair comparison with IDIR, points for training within the lung mask generated by Hofmanninger, J., Prayer, F., Pan, J., Rohrich, S., Prosch, H., Langs, G.: “Automatic lung segmentation in routine imaging is primarily a data diversity problem, not a methodology problem”. European Radiology Experimental 4 (1), 1-13 (2020), were also sampled.

Comparisons with Baseline Methods

Baselines. Six state-of-the-art methods were selected as baselines, including five CNN-based methods (non-INR), and the first INR-based registration method. The six baselines were:
VoxelMorph (Balakrishnan, G., Zhao, A., Sabuncu, M. R., Guttag, J., Dalca, A. V.: Voxelmorph: a learning framework for deformable medical image registration. IEEE transactions on medical imaging 38 (8), 1788-1800 (2019)),
PDD (Heinrich, M. P.: Closing the gap between deep and conventional image registration using probabilistic dense displacement networks. In: MICCAI 2019: Shenzhen, China, Oct. 13-17, 2019, Proceedings, Part VI 22. pp. 50-58. Springer (2019)),
LapIRN (Mok, T. C., Chung, A.: Large deformation diffeomorphic image registration with laplacian pyramid networks. In: International Conference on Medical Image Computing and Computer-Assisted Intervention. pp. 211-221. Springer (2020)),
a CNN-based one-shot registration approach (CNN-Oneshot) (Fechter, T., Baltas, D.: One-shot learning for deformable medical image registration and periodic motion tracking. IEEE TMI 39(7), 2506-2517 (2020)),
VIRNet (Hering, A., H″ager, S., Moltz, J., Lessmann, N., Heldmann, S., van Ginneken, B.: Cnn-based lung ct registration with multiple anatomical constraints. Medical Image Analysis 72, 102139 (2021)),
IDIR (Wolterink, J. M., Zwienenberg, J. C., Brune, C.: Implicit neural representations for deformable image registration. In: International Conference on Medical Imaging with Deep Learning. pp. 1349-1359. PMLR (2022)).

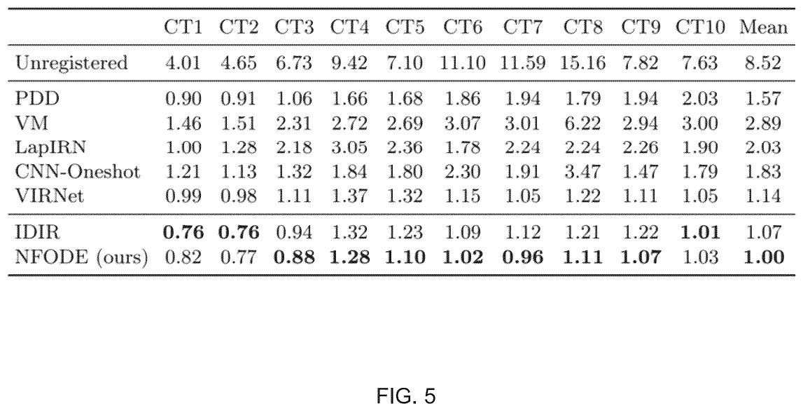

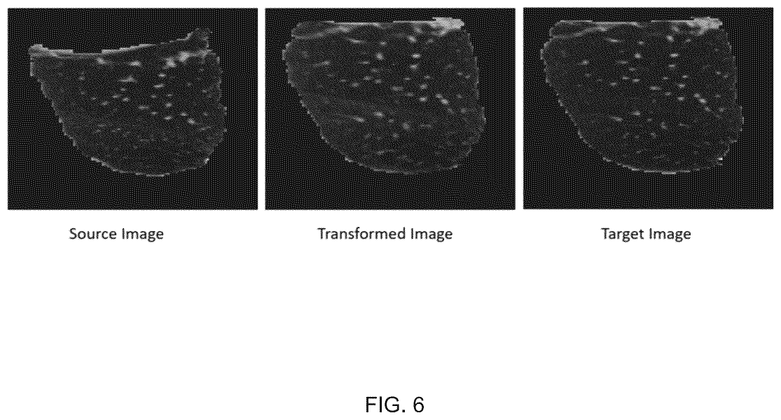

The experimental results are provided in Table 1 (FIG. 5), which presents a comparison of the different methods on DIRLab 4D CT data, with the TRE (mm) of each method on different cases reported. As shown in Table 1, the implementation presented above, referred to as NFODE, achieves the best overall performance compared with both non-INR and INR methods. Compared with the non-INR methods, NFODE outperforms in all cases. NFODE also outperforms the recent INR method IDIR in most CT cases. In particular in case 8, which has the largest initial displacement (large deformation), NFODE significantly outperforms IDIR by 0.1 TRE. This indicates that the proposed approach has a superior advantage in modeling large deformation. FIG. 6 presents the registration results on case 8 of the DIR-LAB dataset, showing slices from inspiration (left), expiration (middle), and transformed images. The visualizations in FIG. 6 also qualitatively demonstrate that NFODE registers the large deformation well.

### Ablation Study

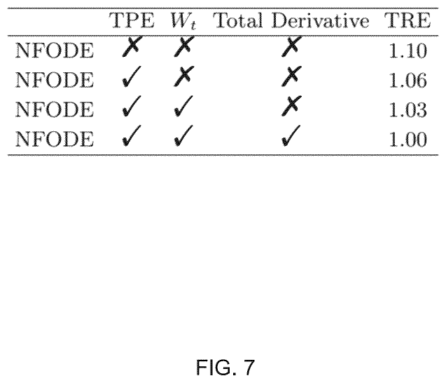

Table 2 (FIG. 7) presents the results of ablation studies on different components of the example implementation to show the effectiveness of, respectively: the proposed time-varying positional encoding (TPE), time-dependent residual weight matrix Wt, and the total derivative regularization. The improvement by TPE and Wt indicates that the non-stationary ODE has a better capability of modeling complex deformation than the stationary one (without either component). The performance gain by the total derivative regularization implies the benefit of having straight-line trajectories (theoretically smoother).

### Influence of Regularization

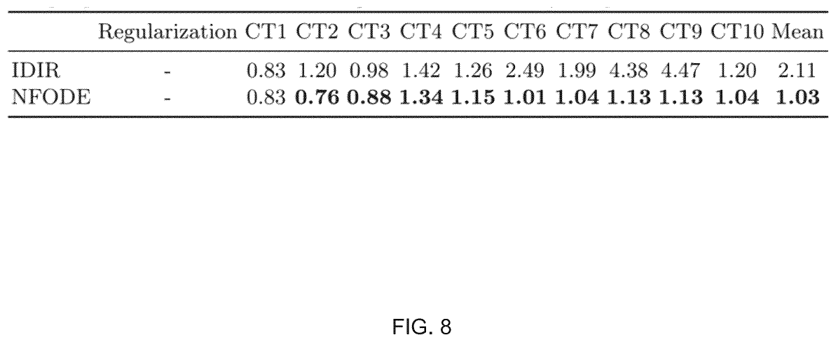

As the example implementation NFODE learns a diffeomorphic field, it can achieve good registration without extra regularization terms, unlike IDIR. Table 3 (FIG. 8) presents the results of an ablation study on the regularization term of IDIR and of NFODE. In Table 3, “BE” indicates the second-order derivative bending energy constraint, and “TD” refers to the proposed total derivative regularization according to examples of the present disclosure. As shown in Table 3, IDIR has a dramatic performance drop by 1.1 TRE without bending energy regularization. In contrast, NFODE retains state-of-the-art performance with only a 0.3 drop in TRE without any regularization. It will be appreciated that NFODE without regularization still outperforms IDIR with bending energy regularization by 0.4 TRE, with three times faster speed. These results demonstrate that the diffeomorphic solution of the ODE is suitable for modeling deformation. Moreover, with total derivative regularization, the performance of NFODE is further improved.

Examples of the present disclosure thus provide methods and nodes that achieve improved performance in the important task of Deformable Image Registration for medical image analysis. Recent advancements using implicit neural representations (INR) have achieved outstanding performance with flexibility in implementing higher-order derivative regularizations, but are limited by their computational complexity and model capacity. The methods disclosed herein increase modeling capability with only minimal reliance on sophisticated regularization. Example methods disclosed herein use a non-stationary ODE parameterized by an implicit neural field for deformable medical image registration. Owing to the diffeomorphic ODE solution, these methods outperform previous models without special regularization. Some examples use time-varying weights for non-stationary ODEs, resulting in larger model capacity and greater flexibility for learning deformation fields. Position encodings with time-varying frequencies, from low to high frequency, allow a coarse-to-fine learning of the deformation field. The dynamic position encoding scheme and time-dependent residual weights enhance the non-stationarity of the methods proposed herein, significantly boosting the model's capacity and adaptability in capturing complex deformation fields. This enables a more robust and flexible learning mechanism for deformation fields. Also, by adopting position encodings that vary in frequency over time, transitioning from lower to higher frequencies, a gradual and detailed learning process of the deformation fields can be achieved. In addition, a total derivative regularization-based regularization term (first derivative) may be used to further smooth the trajectory.

The methods of the present disclosure may be implemented in hardware, or as software modules running on one or more processors. The methods may also be carried out according to the instructions of a computer program, and the present disclosure also provides a computer readable medium having stored thereon a program for carrying out any of the methods described herein. A computer program embodying the disclosure may be stored on a computer readable medium, or it could, for example, be in the form of a signal such as a downloadable data signal provided from an Internet website, or it could be in any other form.

It should be noted that the above-mentioned examples illustrate rather than limit the disclosure, and that those skilled in the art will be able to design many alternative embodiments without departing from the scope of the appended claims or numbered embodiments. The word “comprising” does not exclude the presence of elements or steps other than those listed in a claim or embodiment, “α” or “an” does not exclude a plurality, and a single processor or other unit may fulfil the functions of several units recited in the claims or numbered embodiments. Any reference signs in the claims or numbered embodiments shall not be construed so as to limit their scope.

What is claimed is:

1. A computer-implemented method for performing deformable image registration of a first volumetric medical image associated with a first time instance to a second volumetric medical image associated with a second time instance, the computer-implemented method comprising:
estimating a time varying velocity field between the first volumetric medical image and the second volumetric medical image by, for positions within the first volumetric medical image, and for time instances between the first time instance and the second time instance:
encoding coordinates of the position using a time varying positional embedding; and
using the encoded coordinates and a Neural Field Ordinary Differential Equation (NFODE) to generate a prediction of a rate of change of the deformation field from the first volumetric medical image to the second volumetric medical image;
integrating the estimated velocity field between the first time instance and the second time instance to generate a deformation field from the first volumetric medical image to the second volumetric medical image; and
applying the generated deformation field to the first volumetric medical image to generate a registered volumetric medical image, wherein the NFODE comprises a non-stationary Neural OsummaDE that is parameterized by an Implicit Neural Representation.

2. The computer-implemented method of claim 1, wherein the NFODE comprises a Neural Network that has been trained to approximate an Ordinary Differential Equation.

3. The computer-implemented method of claim 1, wherein the NFODE is implemented as a SIREN network.

4. The computer-implemented method of claim 1, wherein the NFODE comprises a time varying residual weight matrix.

5. The computer-implemented method of claim 1, wherein the NFODE implements:
$$f ( {h}_{i} ) = {σ}_{i} ( {W}_{i} ( t ) {h}_{i} + {b}_{i} ( t ) )$$
$${W}_{i} ( t ) = {W}_{i} + {\sum }_{r = 1}^{{R}_{i}} {c}_{i} ( t ) [ r ] \cdot {M}_{i} [ r ]$$
where: hᵢ∈ [symbol] ^{M}^{ᵢ} is a hidden states at layer i
σᵢ is a sinusoidal activation function
Wᵢ(t) is a time varying residual weight matrix at layer i
bᵢ∈ [symbol] ^{N}^{ᵢ} is a basis vector at layer i
c(t)∈ [symbol] ^{R}^{ᵢ} are trainable coefficients
M∈ [symbol] ^{R}^{ᵢ}^{×N}^{ᵢ}^{×M}^{ᵢ} is a spanning basis set

6. The computer-implemented method of claim 1, wherein the time varying positional embedding comprises a sinusoidal function in which a frequency of the sinusoidal function is time dependent.

7. The computer-implemented method of claim 6, wherein the time varying positional embedding comprises:
$$Γ ( t , p ) = [ sin ( B ( t ) p ) , cos ( B ( t ) p ) ]$$
where: Γ(t, p)∈ [symbol] ⁶ is a time dependent position encoding function
B(t)=2^{-α+βt} controls the frequency of the sinusoidal function and
α and β are hyperparameters

8. The computer-implemented method of claim 1, further comprising, during a training period:
comparing the registered volumetric medical image to the second volumetric medical image; and
updating one or more trainable parameters of the NFODE based at least in part on comparing the registered volumetric medical image to the second volumetric medical image.

9. The computer-implemented method of claim 8, further comprising:
repeating the computer-implemented method of claim 1 using the updated values of the trainable parameters of the NFODE.

10. The computer-implemented method of claim 8, wherein comparing the registered volumetric medical image to the second volumetric medical image comprises calculating a similarity loss between the registered volumetric medical image and the second volumetric medical image.

11. The computer-implemented method of claim 10, wherein the similarity loss comprises Normalized Cross Correlation, NCC, loss.

12. The computer-implemented method of claim 10, wherein comparing the registered volumetric medical image to the second volumetric medical image further comprises calculating a regularization loss.

13. The computer-implemented method of claim 12, wherein the regularization loss comprises a total first order time derivative of a function modelled by the NFODE.

14. The computer-implemented method of claim 12, wherein the regularization loss comprises:
$${L}_{r e g} = {\int }_{0}^{1} {‖ \frac{\partial f ( ϕ , t )}{\partial ϕ} f ( ϕ , t ) + \frac{\partial f ( ϕ , t )}{\partial t} ‖}_{2}^{2} dt$$

15. A computer-implemented method for adaptation of a reference radiotherapy treatment plan, wherein the reference radiotherapy treatment plan is associated with a first volumetric medical image of a patient, the computer-implemented method comprising:
acquiring a second volumetric medical image of a patient;
performing deformable image registration of the first volumetric medical image to the second volumetric medical image by:
estimating a time varying velocity field between the first volumetric medical image and the second volumetric medical image by, for positions within the first volumetric medical image, and for time instances between the first time instance and the second time instance:
encoding coordinates of the position using a time varying positional embedding; and
using the encoded coordinates and a Neural Field Ordinary Differential Equation (NFODE) to generate a prediction of a rate of change of the deformation field from the first volumetric medical image to the second volumetric medical image;
integrating the estimated velocity field between the first time instance and the second time instance to generate a deformation field from the first volumetric medical image to the second volumetric medical image; and
applying the generated deformation field to the first volumetric medical image to generate a registered volumetric medical image, wherein the NFODE comprises a non-stationary Neural OsummaDE that is parameterized by an Implicit Neural Representation; and
using the generated deformation field between the first and second volumetric medical images to adapt the reference radiotherapy treatment plan.

16. A registration node for performing deformable image registration of a first volumetric medical image, associated with a first time instance, to a second volumetric medical image, associated with a second time instance, the registration node comprising processing circuitry configured to cause the registration node to:
estimate a time varying velocity field between the first volumetric medical image and the second volumetric medical image by, for positions within the first volumetric medical image, and for time instances between the first time instance and the second time instance:
encoding coordinates of the position using a time varying positional embedding; and
using the encoded coordinates and a Neural Field Ordinary Differential Equation (NFODE) to generate a prediction of a rate of change of the deformation field from the first volumetric medical image to the second volumetric medical image;
integrate the estimated velocity field between the first time instance and the second time instance to generate a deformation field from the first volumetric medical image to the second volumetric medical image; and
apply the generated deformation field to the first volumetric medical image to generate a registered volumetric medical image, wherein an NFODE comprises a non-stationary Neural ODE that is parameterized by an Implicit Neural Representation.

17. The registration node of claim 16, wherein the registration node is included in a radiotherapy treatment apparatus.

18. A planning node for adapting a reference Radiotherapy, RT, treatment plan, wherein the reference RT treatment plan is associated with a first volumetric medical image of a patient, the planning node comprising processing circuitry configured to cause the planning node to:
acquire a second volumetric medical image of a patient;
perform deformable image registration of the first volumetric medical image to the second volumetric medical image by: estimating a time varying velocity field between the first volumetric medical image and the second volumetric medical image by, for positions within the first volumetric medical image, and for time instances between the first time instance and the second time instance:
encoding coordinates of the position using a time varying positional embedding; and
using the encoded coordinates and a Neural Field Ordinary Differential Equation (NFODE) to generate a prediction of a rate of change of the deformation field from the first volumetric medical image to the second volumetric medical image;
integrating the estimated velocity field between the first time instance and the second time instance to generate a deformation field from the first volumetric medical image to the second volumetric medical image; and
applying the generated deformation field to the first volumetric medical image to generate a registered volumetric medical image, wherein the NFODE comprises a non-stationary Neural OsummaDE that is parameterized by an Implicit Neural Representation; and
use the generated deformation field between the first and second volumetric medical images to adapt the reference treatment plan.

19. The planning node of claim 18, wherein the planning node is included in a radiotherapy treatment apparatus.
